(19) United States
(12) Patent Application Publication
(10) Pub. No.: US 2003/0059877 A1
(43) Pub. Date: Mar. 27, 2003

(54) Human cystine knot polypeptide

(76) Inventors: Sietse Mosselman, Oss (NL); Petrus Johannes Spek Van Der, Lille (BE)

Correspondence Address: William M Blackstone, Akzo Nobel, Patent Department Intervet, 405 State Street, Millsboro, DE, 19966, US

(21) Appl. No.: 10/181,836

(22) PCT Filed: Jan. 17, 2001
(86) PCT No.: PCT/EP01/00570

(30) Foreign Application Priority Data
Jan. 18, 2000 (EP) 00200185.7

(51) Int. Cl. C07K 14/59; C07H 21/04; C12N 15/63; C12P 21/02; C12N 5/06

(52) U.S. Cl. 435/69.1; 435/320.1; 435/325; 530/350; 536/23.5; 514/12

(57) ABSTRACT

The invention relates to newly identified DNA sequences which code for a novel cystine knot polypeptide as well as to the encoded protein. The invention is useful in the field of fertility.

The invention relates to a polynucleotide encoding a novel polypeptide, the protein encoded by that polynucleotide as well as a recombinant cell expressing this protein.

Follicle Stimulating Hormone (FSH), Luteinizing Hormone (LH) and Thyroid Stimulating Hormone from the pituitary, and human chorionic gonadotrophin (hCG) from the placenta belong to the family of glycoprotein hormones. These hormones have a heterodimeric structure, and contain two non-covalently linked α and β subunits. The amino acid sequence of the α subunits is identical, whereas the β subunits differ and confer biological specificity on the individual gonadotrophins (Ulloa-Aquirre, 1988, 1995). Dimers are found to be biologically active. Both the α and β subunits are glycosylated and contain N-linked carbohydrate chains. HCG contains four additional O-linked carbohydrates on the C-terminal peptide.

FSH, LH and TSH are present in most vertebrate species and are synthesized and secreted by the pituitary. CG has so far been found only in primates, including humans, and in horses and is synthesized by placental tissue.

Within a species, the α-subunit is essentially identical for each member of the glycoprotein hormone family; it is also highly conserved from species to species. The β-subunits are different for each member, i.e. CG, FSH, TSH and LH, but show considerable homology in structure. Furthermore, also the β subunits are highly conserved from species to species. In humans, the mature α subunit consists of 92 amino acid residues, whilst the β subunit varies in size for each member: 111 residues in hFSH, 121 residues in hLH, 118 residues in hTSH and 145 residues in hCG (Combarnous, Y. (1992), Endocrine Reviews, 13, 670-691, Lustbader, J. W. et al. (1993), Endocrine Reviews, 14, 291-311). The β subunit of hCG is substantially larger than the other β subunits in that it contains 34 additional amino acids at the C-terminus referred to herein as the carboxy terminal protein (CTP).

The two subunits of the heterodimer display many conserved intra-subunit disulfide bonds: five disulfide bridges in the α-subunit and six disulfide bridges in the β-subunit. The corresponding cystein residues are fully conserved among all members of the gonadotropin family. In the β subunit of hCG the disulfide bridges are formed between the cysteins at positions 9-57; 23-72, 26-110, 34-88, 38-90 and 93-100. The X-ray structure of hCG shows that these disulfide bonds are involved in typical three-dimensional patterns called disulfide knots. The hormones possess three or four asparagine residues that can be N-glycosylated. In addition, the C-terminal peptide (CTP) of hCG can be O-glycosylated at four serine positions.

The glycoprotein hormones serve important functions in a variety of bodily functions including metabolism, temperature regulation and the reproductive process. The pituitary gonadotropin FSH for example plays a pivotal role in the stimulation of follicle development and maturation, whereas LH induces ovulation (Sharp, R. M. (1990), Clin Endocrinol., 33, 787-807; Dorrington and Armstrong (1979), Recent Prog. Horm. Res., 35, 301-342). Currently, FSH is applied clinically, either alone or in combination with LH activity, for ovarian stimulation i.e. ovarian hyperstimulation for in vitro fertilization (IVF) and induction of in vivo ovulation in infertile anovulatory women (Insler, V.(1988), Int. J. Fertility, 33, 85-97, Navot and Rosenwaks (1988), J. Vitro Fert. Embryo Transfer, 5, 3-13), as well as for male hypogonadism. The aim of controlled superovulation is to increase the number of retrievable mature oocytes for IVF and subsequent embryo transfer (ET). Generally, up to three embryos are replaced per transfer. As usually more than one treatment is necessary, in most infertility clinics spare embryos or fertilized oocytes are frozen and transferred in subsequent cycles.

TSH can be used by patients in need for thyroid hormone supplements e.g. for use in thyroid cancer patients who have had partial or total removal of their thyroid gland.

Genomic and cDNA clones have been prepared for all subunits and their primary structure has been resolved. Moreover, Chinese Hamster Ovary (CHO) cells have been transfected with human gonadotropin subunit genes and these cells are shown to be capable of secreting intact dimers (e.g. Keene et al (1989), J.Biol.Chem., 264, 4769-4775; Van Wezenbeek et al (1990), in From clone to Clinic (eds Crommelin D. J. A. and Schellekens H., 245-251).

In principle, the regulation of fertility can be influenced at several stages e.g. follicle recruitment, folliculogenesis, implantation and maintenance of pregnancy.

Due to selection mechanisms only one follicle from the group of follicles that left the primordial pool, reaches the preovulatory stage, i.e. the dominant follicle, and provides a healthy, fertilizable oocyte. The others become atretic and degenerate. The mechanisms controlling the selection of a dominant follicle are not fully understood, but it has been hypothesized that the follicle most sensitive to FSH is the one that becomes dominant. It is well known that in addition to gonadotropins other factors are needed for optimal follicle and oocyte development. Follicular growth is controlled by growth factors such as IGF-1 and GDF-9, and at later stages by the gonadotropins FSH and LH, and by estrogens. Regulatory factors are also involved in the control of follicular arrest, early follicular recruitment, follicular growth, anthral formation and the process of ovulation. Also, these regulatory factors influence the process of embryo implantation in the uterus and are involved in regulation of spermatogenesis in the male.

There is a need to identify factors involved in different stages of female and male fertility. Such factors can be used in either in vivo or in vitro therapeutic protocols.

The present invention provides for such a factor. More specific, the present invention provides for a polynucleotide sequence comprising encoding SEQ ID NO:1.

The complete genetic sequence can be used in the preparation of vector molecules for expression of the protein factor in suitable host cells. Complete genes or variants thereof can be derived from cDNA or genomic DNA from natural sources or synthesized using known methods.

The invention also includes the entire mRNA sequence as indicated in SEQ ID NO:1. The mRNA contains an open reading frame corresponding to nucleotide sequence 101-490 of SEQ ID NO:1. This sequence encodes a precursor protein of 130 amino acids (SEQ ID NO:2). Furthermore, to accommodate codon variability, the invention also includes sequences coding for the same amino acid sequences as the sequences disclosed herein. Also portions of the coding sequences coding for a functional polypeptide are part of the invention as well as allelic and species variations thereof. Sometimes, a gene is expressed in a certain tissue as a splicing variant, resulting in an altered 5′ or 3′ mRNA or the inclusion or exclusion of one or more exon sequences. These sequences as well as the proteins encoded by these sequences all are expected to perform the same or similar functions and form also part of the invention.

In particular, SEQ ID NO:3 represents a specific splice variant which differs from SEQ ID NO:1 in that an insertion of 128 nucleotides is present. Translation of this splice variant leads to a truncated version of the protein in SEQ ID NO:2, as shown in SEQ ID NO:4.

It has now been found that these sequences specifically are expressed in pituitary and endometrium.

The sequence information as provided herein should not be so narrowly construed as to require exclusion of erroneously identified bases. The specific sequence disclosed herein can be readily used to isolate the complete genes of several species.

Thus, in one aspect, the present invention provides for isolated polynucleotides encoding a novel protein hormone.

The term isolated denotes that the polynucleotide has been removed from its natural environment and is thus in a form suitable for use within genetically engineered protein production systems.

The DNA according to the invention may be obtained from cDNA. The tissues preferably are from human origin. Preferably ribonucleic acids are isolated from pituitary, placenta or endometrium. Alternatively, the coding sequence might be genomic DNA, or prepared using DNA synthesis techniques. The polynucleotide may also be in the form of RNA. If the polynucleotide is DNA, it may be in single stranded or double stranded form. The single strand might be the coding strand or the non-coding (anti-sense) strand.

The polypeptide according to the present invention can exist as a monomer. However, also dimeric forms of the peptide are part of the invention. Such dimers are homodimers consisting of two identical polypeptides or, as an alternative, heterodimer complexes. Preferably such a dimer consists of the polypeptide according to the invention combined with the common α subunit of the gonadotropin hormone family. As an alternative also chimeric proteins are envisaged comprising the functional part of the sequence of the polypeptide according to the invention. Such chimeric construct can easily be prepared by linking the DNA encoding the subunits of the heterodimeric complex joined by a linker as described in PCT application WO96/05224. Similarly, bifunctional glycoproteins can be prepared wherein the subunit of the present invention is joined covalently by linkers to other members of the glycoprotein hormone family. Examples of such constructs are described in PCT application WO99/25849.

The present invention further relates to polynucleotides having slight variations or having polymorphic sites. Polynucleotides having slight variations encode polypeptides which retain the same biological function or activity as the natural, mature protein. Polymorpic sites are useful for diagnostic purposes.

Such polynucleotides can be identified by hybridization under preferably highly stringent conditions. According to the present invention the term “stringent” means washing conditions of 1×SSC, 0.1% SDS at a temperature of 65° C.; highly stringent conditions refer to a reduction in SSC towards 0.3×SSC, more preferably to 0.1×SSC. Preferably the first two washings are subsequently carried out twice each during 15-30 minutes. If there is a need to wash under highly stringent conditions an additional wash with 0.1×SSC is performed once during 15 minutes. Hybridization can be performed e.g. overnight in 0,5M phosphate buffer pH7.5/7% SDS at 65° C.

Thus, also functional equivalents that is polypeptides comprising SEQ ID NO:1 or parts thereof having variations of the sequence while still maintaining functional characteristics, are included in the invention.

The DNA according to the invention will be very useful for in vivo or in vitro expression of the novel protein according to the invention in sufficient quantities and in substantially pure form.

The variations that can occur in a sequence may be demonstrated by (an) amino acid difference(s) in the overall sequence or by deletions, substitutions, insertions, inversions or additions of (an) amino acid(s) in said sequence. Amino acid substitutions that are expected not to essentially alter biological and immunological activities, have been described. Amino acid replacements between related amino acids or replacements which have occurred frequently in evolution are, inter alia Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Based on this information Lipman and Pearson developed a method for rapid and sensitive protein comparison (Science, 1985, 227, 1435-1441) and determining the functional similarity between homologous polypeptides. It will be clear that also polynucleotides coding for such variants are part of the invention.

Thus, in another aspect of the invention there are provided polypeptides comprising SEQ ID NO:2 or SEQ ID NO:4 but also polypeptides with a similarity of 70%, preferably 90%, more preferably 95%, even more preferably 98%. NCBI-BLASTX 2.0.4 [Feb. 24, 1998] (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) is used to search for sequence alignments using default settings. For amino acid alignments the BLOSUM62 matrix is used as a default and the similarity is indicated as the number of positives. No filtering of low compositional complexity is included.

Preferably, the polypeptide comprises cystein residues at positions corresponding to amino acid positions 36, 50, 60 and 64 of SEQ ID NO:2 or SEQ ID NO:4. Even more preferably cystein residues are present at positions corresponding to amino acid positions 84, 99, 115, 117, 120, and 127 of SEQ ID NO:2. Corresponding to a certain position indicates the position in a second sequence that aligns with the reference sequence as indicated in SEQ ID NO:2 or SEQ ID NO:4 when the sequences are optimally aligned. Thus the polypeptide is capable of forming all disulphide bridges at the corresponding positions as compared to the 13 subunit of the glycoprotein hormone family with the exception of the so-called seat belt disulphide bond (at corresponing positions 26-110 of β hCG).

The protein as indicated in SEQ ID NO:2 or SEQ ID NO:4 is a precursor protein and is subjected during secretion to a proteolytic cleavage. The mature proteins are also part of the invention. The protein as indicated in SEQ ID NO:2 or SEQ ID NO:4 as well as the mature protein may be subject to post-translational modifications, for instance glycosylation. Such modified proteins are also part of the invention.

It is to be understood that also portions of such polypeptides still capable of conferring biological effects are included. Especially portions which still bind to targets form part of the invention. Such proteins or functional parts thereof may be functional per se, e.g. in solubilized form or they may be linked to other polypeptides (e.g. CTP, WO90/09800), either by known biotechnological ways or by chemical synthesis, to obtain chimeric proteins. Such proteins might also be useful as therapeutic agent by preventing the target from interacting with the natural proteins in the body. Thus, such altered proteins might be used as an agonist or an antagonist of its natural function. In this respect also antibodies against the protein according to the invention form part of the invention. Such antibodies can be prepared by conventional hybridoma technology or recombinant DNA technologies (Antibodies, A laboratory manual, 1988, Cold Spring Harbor Laboratory).

Alternatively, downregulation of the expression level of the protein can be obtained by using anti-sense nucleic acids through triple-helix formation (Cooney et al., 1988, Science, 241, 456-459) or by binding to the mRNA. This in itself could also lead to regulation of fertility i.e. contraception or treatment of infertility.

The present invention comprises all isolated polynucleotides which comprise in their coding sequence the polypeptides as indicated above. A wide variety of host cell and cloning vehicle combinations may be usefully employed in cloning the nucleic acid sequence coding for the polypeptide according to the invention.

Suitable expression vectors are for example bacterial or yeast plasmids, wide host range plasmids and vectors derived from combinations of plasmid and phage or virus DNA. Vectors derived from chromosomal DNA are also included. Furthermore an origin of replication and/or a dominant selection marker can be present in the vector according to the invention. The vectors according to the invention are suitable for transforming a host cell.

In case of dimeric proteins similar cloning vehicles may be used for insertion of a second subunit into the host cell. Subunits might be encoded by different vectors as well as by a single vector.

Vehicles for use in expression of the protein or parts thereof of the present invention will further comprise control sequences operably linked to the nucleic acid sequence coding for the protein. Such control sequences generally comprise a promoter sequence and sequences, which regulate and/or enhance expression levels. Of course control and other sequences can vary depending on the host cell selected.

Recombinant expression vectors comprising the DNA of the invention as well as cells transfected with said DNA or said expression vector, either transiently or stable, also form part of the present invention.

Suitable host cells according to the invention are bacterial host cells, yeast and other fungi, plant or animal host such as Chinese Hamster Ovary cells or monkey cells. Thus, a host cell which comprises the DNA or expression vector according to the invention is also within the scope of the invention. The engineered host cells can be cultured in conventional nutrient media which can be modified e.g. for appropriate selection, amplification or induction of transcription. The culture conditions such as temperature, pH, nutrients etc. are well known to those ordinary skilled in the art.

The techniques for the preparation of the DNA or the vector according to the invention as well as the transformation or transfection of a host cell with said DNA or vector are standard and well known in the art, see for instance Sambrook et al., Molecular Cloning: A laboratory Manual. 2nd Ed., Cold Spring Harbor Laboratory, Cold Spring Harbor, N.Y., 1989.

Culturing host cells comprising vectors encoding the polypeptide according to well-known methods and recovering the polypeptide of interest can produce the polypeptide according to the invention. Dimeric proteins can similarly be isolated from culturing cells transfected with an additional vector encoding the second protein or by culturing cells transfected with a single vector encoding both subunits.

The polypeptide according to the invention can be recovered and purified from recombinant cell cultures by common biochemical purification methods (as described in Guide to Protein purification. Edited by Murray P. Deutscher. (1990) Methods in Enzymology.Vol 182. Academic Press, inc. San Diego Calif. 92101. Harcourt Brace Jovanovich, Publischers. including ammonium sulfate precipitation, extraction, chromatography such as hydrophobic interaction chromatography, cation or anion exchange chromatography or affinity chromatography and high performance liquid chromatography. If necessary, also protein refolding steps can be included. Alternatively the protein can be expressed and purified as a fusion protein containing (“tags”) which can be used for affinity purification.

The polypeptide according to the invention is useful for the control of follicular arrest and recruitment. Inhibition of recruitment can be used to delay (premature) menopause or as a contraceptive. In addition, this polypeptide can be employed for in vitro maturation and growth of follicles e.g. from frozen ovarian tissue.

The polypeptides of the invention are also useful in detecting and purifying receptors to which the proteins bind. For instance, the polypeptides may be coupled to solid supports and used in affinity chromatographic preparation of receptors or antihormone antibodies. The receptors are themselves useful in assessing hormone activity for candidate drugs in screening tests for therapeutic candidates. Such candidate drugs might behave as agonists or antagonists of the polypeptide according to the invention and as such might improve the implantation efficiency of embryos or prevent the implantation.

The invention also provides for the formulation of a pharmaceutical composition comprising mixing the protein according to the invention with a pharmaceutically acceptable carrier.

Pharmaceutical acceptable carriers are well known to those skilled in the art and include, for example, sterile saline, lactose, sucrose, calcium phosphate, gelatin, dextrin, agar, pectin, peanut oil, olive oil, sesame oil and water.

Furthermore the pharmaceutical composition according to the invention may comprise one or more stabilizers such as, for example, carbohydrates including sorbitol, mannitol, starch, sucrosedextrin and glucose, proteins such as albumin or casein, and buffers like alkaline phosphates. Methods for making preparations and intravenous admixtures are disclosed in Remingtons's Pharmaceutical Sciences, pp. 1463-1497 (16th ed. 1980, Mack Publ. Co of Easton, Pa., USA). Therapeutical dosages will generally be in the range of 0.1-100 μg/kg of patient weight per day, preferably 0.5-20 μg/kg per day.

Thus, the protein according to the invention is useful in the preparation of a pharmaceutical. The pharmaceutical is to be used in fertility related disorders or in contraception.

### LEGENDS TO THE FIGURES

FIG. 1 RT PCR using primers SEQ ID NO:5 and SEQ ID NO:6 using human pituitary cDNA as a template.

FIG. 2 Alignment of SEQ ID NO:2 with partial sequences derived from monkey, porcine and rabbit, respectively. Dashes indicate that no sequence information is available.

FIG. 3 Overview of a human tissue array section stained with H&E (haematoxilin-eosin).

FIG. 4 In situ hybridization

a. endometrium (secretory phase) section hybridized with antisense probe

b. endometrium (secretory phase) section hybridized with sense probe

c. pituitary (secretory phase) section hybridized with antisense probe

d. endometrium (secretory phase) section hybridized with sense probe

### EXAMPLES

### Example 1

### Sequence Identification

Using parts of the DNA sequence and/or protein sequence of the beta subunit of human FSH (βFSH) we have screened several databases for the presence of related sequences. A human genomic clone was identified which contains a region with a low degree of overall homology. However, the genomic sequence predicted an open reading frame wherein a number of cystein residues were present with a spacing that was very similar to that of βFSH and related proteins like βLH, βhCG and βTSH.

To obtain a DNA fragment corresponding to the novel gene, a PCR on human genomic DNA using primers SEQ ID NO:5 and SEQ ID NO:6 was performed. A fragment with the expected size of 142 base pairs was obtained, cloned into PCR2.1 vector and sequenced. The sequence was identical to part of the genomic clone and corresponds to nucleotide 337 to 478 in SEQ ID NO:1.

In order to clone full-length cDNA encompassing the complete open reading frame (ORF), we performed 5′ and 3′ RACE (rapid amplification of cDNA ends) PCR experiments. As template we used Marathon-ready cDNA derived from human pituitary (Clontech cat # 7424-1). For 5′ RACE, in the first PCR, the AP1 primer (SEQ ID NO:7.) from the kit was used together with the gene-specific primer SEQ ID NO:6 using 5 microliter of pituitary cDNA as template. For 3′ RACE, similarly, the first reaction was performed using primers SEQ ID NO:7 with SEQ ID NO:5. The PCR protocol was as follows: 5 min. 94° C.; 5 cycles 5 sec. 94° C./4 min. 72° C.; 5 cycles 5 sec 94° C./4 min. 70° C.; 25 cycles 5 sec. 94° C./4 min. 68° C.; 5 min. 72° C.; store at 4° C.

Subsequently, nested PCR reactions were performed using 1% of the volume of the first PCR as template. Here, primer AP2 (SEQ ID NO:8) from the kit was used in combination with primer SEQ ID NO:9 for the 5′ RACE. For 3′ RACE primer SEQ ID NO:8 was used in combination with SEQ ID NO:10. The nested reactions were performed using the Advantage 2 cDNA polymerase kit (Clontech) with the following protocol: 5 min. 94° C.; 20 cycles 5 sec. 94° C./4 min. 68° C.; 5 min. 68° C.; storage at 4° C.

PCR products were analysed on 1.2% agarose gel and the gel was overnight blotted in 20×SSC onto Hybond N+ nitrocellulose. DNA was cross-linked by baking for 2 hours at 80° C. The blot was hybridized (overnight at 65° C. in 0,5 molar phosphate buffer pH 7.5/7% SDS) with the 142 base pair gene-specific PCR fragment that is described above. Filters were washed in 0.3×SSC/0.1% SDS at 65° C. and subsequently in 0.1×SSC/0.1% SDS at 65° C. A hybridizing fragment of approximately 480 base pairs originating from the 5′ RACE reaction was cut from the gel, purified using a Qiaquick gel extraction kit (Qiagen) according to the manufacturers instructions. Similarly, a hybridizing band of approximately 650 base pairs was isolated for the 3′ RACE reactions. Both fragments were cloned into pCR2.1 vector and sequenced. The resultant 5′ and 3′ RACE fragments revealed overlapping sequences as expected. The 5′ fragment sequence corresponds to nucleotide 1 to 449 in SEQ ID NO:1. The 3′ fragment sequence corresponds to nucleotide 377 to 917 in SEQ ID NO:1, followed by a stretch of A-residues. The AP2 sequence as well as most of the poly-A stretch are omitted in SEQ ID NO:1.

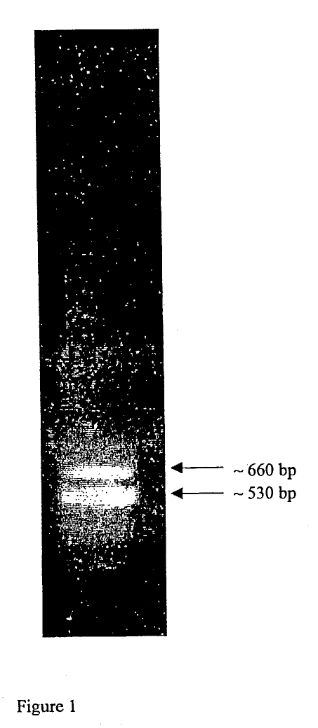

To verify the sequences that were obtained, a PCR was performed to amplify the region encompassing the ORF using two primers: one upstream of the ATG translation initiation codon (SEQ ID NO:11) and the other downstream of the stopcodon (SEQ ID NO:12). An expected fragment of approximately 530 base pairs was obtained as a major band using pituitary cDNA as a template (see FIG. 1). The sequence of this fragment corresponds to nucleotides 23 to 548 of SEQ ID NO:1 and was identical to that of (part of) the combined RACE fragments.

SEQ ID NO:1 contains an open reading frame (nucleotides 101 to 490) coding for 130 amino acids. Upstream of the ATG translation initiation codon an in-frame stopcodon is present (nucleotides 44 to 46). A polyadenylation signal (ATTAAA, nucleotides 894 to 899) is followed somewhat downstream by a poly A stretch, which is only partially included in SEQ ID NO:1. The open reading frame contains 10 cystein residues with a spacing that is extremely similar as it is in βFSH, βLH, βhCG and βTSH. The amino terminal region of the reading frame probably corresponds to a signal sequence. A number of characteristics can be noted e.g. the presence a stretches of hydrophobic residues as well as the presence of a basic amino acid following the amino terminal methionine.

Comparison of the complete sequence of SEQ ID NO:1 with human genomic DNA sequences revealed that the novel gene consists of three exons. Exon 1 corresponds to nucleotides 1 to 99, exon 2 corresponds to nucleotides 100 to 304 and exon 3 corresponds to nucleotides 305 to 911.

FIG. 1 shows that in addition to the expected fragment of approximately 530 base pairs a second fragment is obtained which is somewhat longer (approximately 660 base pairs). This fragment was cloned and sequehced and it was established that it corresponds to a splice variant containing sequences of an intron (corresponding to SEQ ID NO:3). The encoded protein is shown in SEQ ID NO:4.

### Example 2

### Evolutionary Conservation

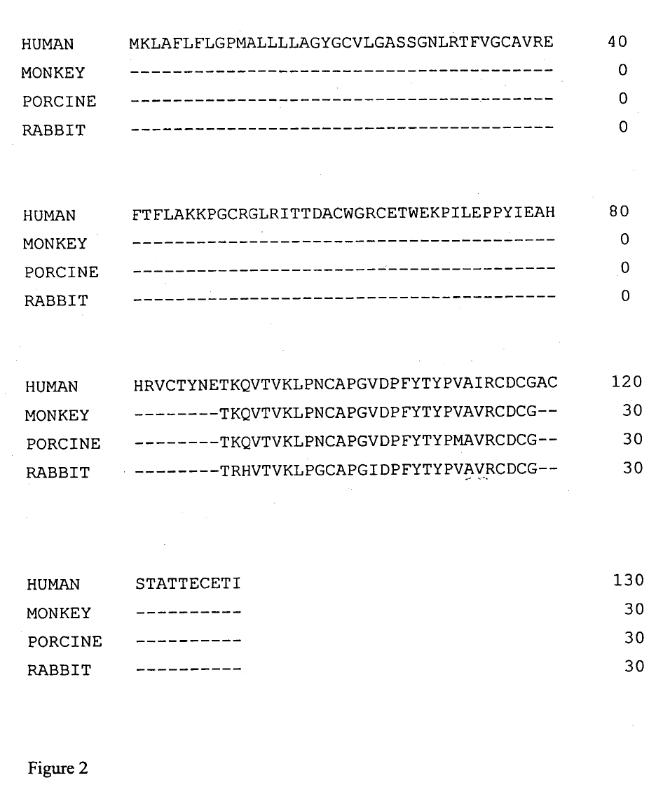

To establish whether the novel gene is conserved in evolution, primers SEQ ID NO:5 and SEQ ID NO:6 were also used for PCR reactions using genomic DNA from pig, monkey and rabbit. Fragments of the expected size were obtained and analysed by cloning the purified fragments in pCR2.1 and nucleotide sequencing. All three sequences are extremely homologous to the human sequence. When the sequences of the primers used for PCR are omitted, an alignment of the deduced amino acid sequences shows a high degree of sequence conservation (see FIG. 2).

### Example 3

### In Situ Hybridization

Human Tissue Arrays

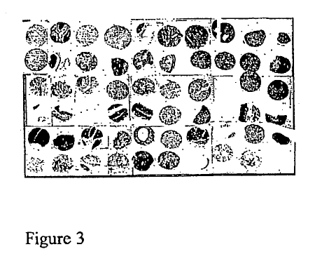

Tissue arrays were obtained from Superbiochips Laboratories (Seoul, Korea, FH-A1 and FH-A2, FIG. 3). In short, the tissue arrays used consisted of 60 different normal human tissue cylinders of 4 mm in diameter. Each cylinder was punched out of a specimen that had been previously fixed in formalin and routinely embedded in paraffin. All 60 cylinders were assembled into one single paraffin block. Then 5 μm sections were cut and collected on RNase-free object slides.

Generation of Sense and Antisense RNA Probes

With specifically designed primer sets containing either a T7 and SP6 RNA polymerase site a unique part of the gene was amplified. Using this approach both sense and antisense probes could be generated from a single PCR fragment. The PCR mixture contained SP6 forward primer (2 ng/μl) (SEQ ID NO:13), T7 reverse primer (2 ng/μl) (SEQ ID NO:14), 1×PCR buffer (Pharmacia, with 15 mM MgCl2), dNTP mix (0.2 mM/dNTP), Taq polymerase (0.02 U/μl) and DNA template (0.5 ng/μl). The PCR reaction consisted of initial denaturation (5 min 95° C.), 8 cycles at a low annealing temperature (0.5 min 95° C., 0.5 min 55° C., 1 min 72° C.), and 30 cycles at a high annealing temperature (0.5 min 95° C., 0.5 min 60° C., 1 min 72° C.), and 5 min at 72° C. 5-10 μl of PCR product was run on a 2% agarose gel to confirm the yield and correct amplification of the expected DNA fragment. The PCR product was ethanol precipitated overnight, centrifuged (14,000 rpm), washed in 70% ethanol and subsequently resuspended in H₂O. After purification on GFX columns (Pharmacia) the concentration of the probe was calculated based on OD260/OD280 values and diluted to a final concentration of 100 ng/μl.

RNA probes were generated starting with 500 ng of template (according the the protocol provided by the manufacturer, Boehringer-Roche) in the presence of DIG labeling mix (DIG-UTP, unlabeled nucleotides, blocking agents), transcription buffer, 10 mM DTT, 1 U/μl RNase inhibitor and 2-4 U/μl the proper RNA polymerase. Incubations were performed at 37° C. for 2 hrs and stopped by adding approximately 25 mM EDTA (pH 8.0), 400 mM LiCl and excess of 100% ethanol. The labeled product was precipitated overnight, centrifuged, washed in 70% ethanol and subsequently resuspended in H₂O with RNase inhibitor. After in vitro transcription a small amount of the probe was analyzed on a 1.5% agarose gel to confirm successful in vitro transcription. Probe concentrations were estimated according to a Boehringer-Roche protocol and using the advised reagents. Serial dilutions of labeled probe and control DIG-RNA of known concentration (10-0.01 ng/μl) were spotted on a Hybond N+ (Amersham) membrane. The membrane was microwaved for 2 min. After blocking for aspecific binding the membrane was incubated with anti-DIG alkaline phosphatase Fab′ fragments (anti-DIG-AP) for 30 min. Staining was started by adding NBT/BCIP substrate and continued until sufficient staining was seen in the lowest concentration of the control series. The concentration of the freshly labeled probe was estimated by comparing the intensity of the dot-spots with those of the control series.

In situ Hybridization

Tissue sections were baked at 60° C. for two hours, dewaxed in xylene en rehydrated in decreasing concentrations of ethanol. Subsequently the sections were treated for 20 min in 0.2M HCl, washed in DEPC treated Milli Q. and digested with proteinase K (1 μg/ml) in digest buffer (100 mM Tris, 50 mM EDTA pH 8) for 30 min at 37° C. Digestion was stopped in prechilled 0.2% glycine in PBS for 10 min at room temperature (RT). The slides were acetylated for 5 min with 0.25% acetic anhydride in 0.1 M triethanolamine buffer, followed by two washes in DEPC treated Milli Q. Sections were prehybridized at hybridization temperature in a humid chamber with prehybridization mix, containing 52% formamide, 21 mM Tris, 1 mM EDTA, 0.33 M NaCl, 10% dextran sulfate, 1×Denhardt's solution, 100 μg/ml salmon sperm DNA, 100 μg/ml tRNA and 250 μg/ml yeast total RNA. The slides were covered with a glass coverslip. After two hours prehybridization mix was replaced with probe hybridization mix containing prehybridisation mix with the following additions: 0.1 mM DTT, 0.1% sodium thiosulphate, 0.1% SDS and a varying amount of DIG-labeled probe. The hybridization was carried out overnight (16 hours) in a humid chamber at 50° C.

Slides were then washed in 2×SSC for 15 min, followed by washes in 2×SSC, 1×SSC and 0.1×SSC each for 15 min at hybridization temperature. Sections were treated with Ribonuclease A (20 μg/ml) in RNase buffer (0.6 M NaCl, 20 mM Tris, 10 mM EDTA) for 1 hour at 37° C. After two washes (5 min RT) in prechilled PBS and one wash in buffer 1 (100 mM maleic acid, 150 mM NaCl), the sections were incubated for 30 min with blocking solution (1 g/ml blocking reagent in buffer 1). Then the sections were incubated with anti-DIG-AP (Boehringer/Roche), diluted 1:500 in blocking solution, for 1 hour at RT. After two washes in buffer 1 (15 min RT) the slides were carefully wiped dry around the tissue and the sections were encircled with a DAKO-pen® (DAKO). The sections were covered with NBT/BCIP color development reagent (Boehringer/Roche) and incubated in a hurnid chamber at RT. After two hours the sections were rinsed in water and optionally counterstained with 0.1% methyl green for 30 seconds. Slides were mounted in Kaiser's glycerol-gelatin.

In all experiments both antisense and sense probes were used at different concentrations (200 and 1000 ng/ml). The hybridization temperatures used was 50° C.

Microscopic Evaluation

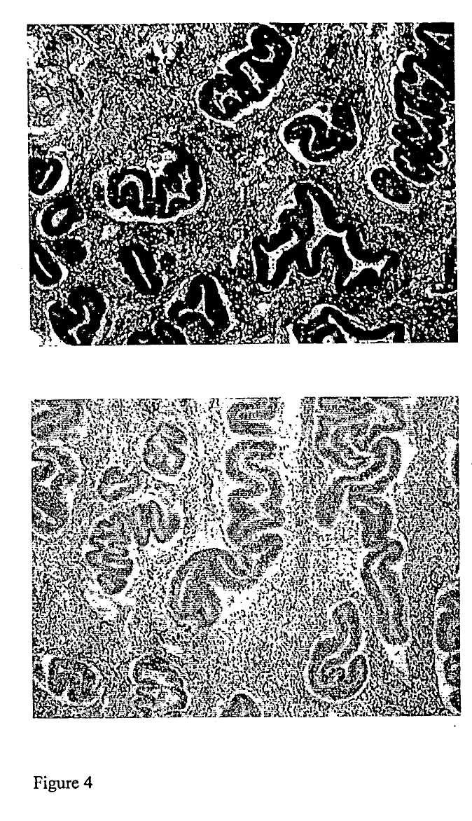

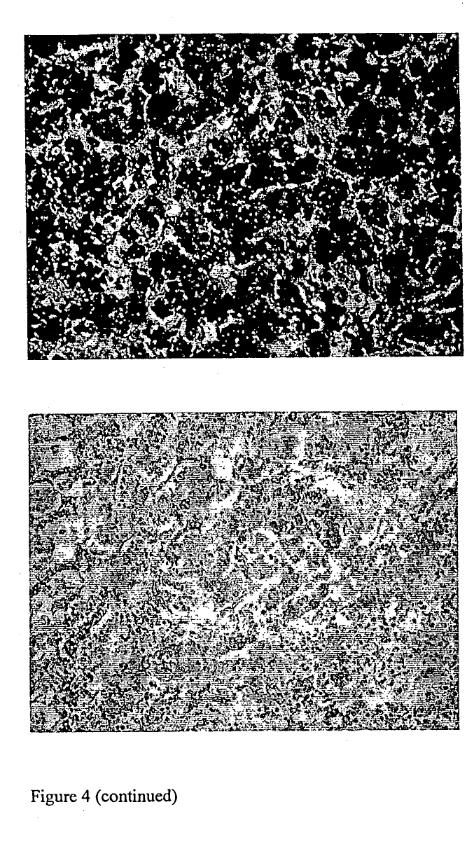

The in situ hybridization analysis revealed that two tissues showed significant staining with the antisense probe as compared to the sense probe. These tissues were endometrium (see FIG. 4a and 4b) and pituitary (see FIG. 4c and 4d). All other tissues were negative.

1. An isolated polynucleotide encoding a polypeptide that is at least 70% similar to SEQ ID NO:2 or SEQ ID NO:4.

2. An isolated polynucleotide encoding a mature polypeptide that is at least 70% similar to the mature polypeptide part of SEQ ID NO:2 or SEQ ID NO:4.

3. The polynucleotide of claim 1 or 2 which is at least 90, preferably 95% similar to SEQ ID NO:2 or SEQ ID NO:4.

4. The polynucleotide of claims 1-3 said polypeptide comprising the amino acid Cys at positions corresponding to amino acid positions 36, 50, 60 and 64 of SEQ ID NO:2 or SEQ ID NO:4.

5. The polynucleotide of claim 4 with the amino acid Cys at positions corresponding to amino acid positions 84, 99, 115, 117, 120 and 127 of SEQ ID NO:2.

6. The polynucleotide according to claim 5, said polynucleotide comprising the sequence SEQ ID NO:1 or the sequence extending from nucleotides 101-490 of SEQ ID NO:1.

7. The polynucleotide according to claim 4, said polynucleotide comprising the sequence SEQ ID NO:3 or the sequence extending from nucleotides 101-325 of SEQ ID NO:3.

8. A recombinant expression vector comprising the DNA according to claims 1-7.

9. Polypeptide encoded by the polynucleotide according to claims 1-7 or the expression vector according to claim 8.

10. A cell transfected with DNA according to claims 1-7 or the expression vector according to claim 8.

11. A cell according to claim 10 that is a transfected cell that expresses the protein according to claim 9.

12. A method to produce the polypeptide of claim 9 the method comprising culturing the cells of claim 11 under conditions wherein said protein is produced and recovering said protein from the culture.

13. A pharmaceutical composition comprising a polypeptide according to claim 9 in admixture with a pharmaceutically acceptable carrier.


SEQUENCE LISTING (14 sequences)

SEQ ID NO: 1 - DNA, 917 bp, Homo sapiens
gacatttacccagggcaaacttctaccattcattgtgacttcctgaaatcttagtgcaagtttcagctctaaaagaagagtgggctcctgcaagattagcatgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggccgctagctgctctctgcagacccgcctgtgtgagcagcacatgcagttatacttcctggatgcaagactgtttaatttcgaccacacccatggaggaggttacctgtcgccccttaggtccagctcaggcaaaaggcccaaatgcagcctacttatgctaaaagttcaaaacaatattcgtgccttcaccaaaataatttctccagctcacatacctgcaaattaatttttctttgccttgagtcttggaacataatttgtgtatcacaatcctcccccaatttggacttataatatgctaatgatttaaacacatgggatgtaattaggatatggggctggaaagtctttaaattctcatgttctatttaacctctgatctccaaccggatttatgattaaagggctagaaatgaaaaaa

SEQ ID NO: 2 - protein, 128 aa, Homo sapiens
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTTPVAIRCDCGACSTATTECE

SEQ ID NO: 3 - DNA, 1045 bp, Homo sapiens
gacatttacccagggcaaacttctaccattcattgtgacttcctgaaatcttagtgcaagtttcagctctaaaagaagagtgggctcctgcaagattagcatgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagcttttgtcaagatgtcgtgtatgaacaaggcattcaatacacatttgttggttgactgggatggacctccccctggagctgtagatcctccagcctaatggaaggccatttagaatcacacttgcactaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggccgctagctgctctctgcagacccgcctgtgtgagcagcacatgcagttatacttcctggatgcaagactgtttaatttcgaccacacccatggaggaggttacctgtcgccccttaggtccagctcaggcaaaaggcccaaatgcagcctacttatgctaaaagttcaaaacaatattcgtgccttcaccaaaataatttctccagctcacatacctgcaaattaatttttctttgccttgagtcttggaacataatttgtgtatcacaatcctcccccaatttggacttataatatgctaatgatttaaacacatgggatgtaattaggatatggggctggaaagtctttaaattctcatgttctatttaacctctgatctccaaccggatttatgattaaagggctagaaatgaaaaaa

SEQ ID NO: 4 - protein, 75 aa, Homo sapiens
MKLAFLFLGPMALLLLAGYGCVLGASSGNLRTFVGCAVREFTFLAKKPGCRGLRITTDACWGRCETWELLSRCRV

SEQ ID NO: 5 - DNA, 26 bp, Homo sapiens
ccatcatcgagtctgtacctacaacg

SEQ ID NO: 6 - DNA, 23 bp, Homo sapiens
ctccgtggtggcagtggagcagg

SEQ ID NO: 7 - DNA, 27 bp, Homo sapiens
ccatcctaatacgactcactatagggc

SEQ ID NO: 8 - DNA, 23 bp, Homo sapiens
actcactatagggctcgagcggc

SEQ ID NO: 9 - DNA, 25 bp, Homo sapiens
agtcacagcggatggccacgggata

SEQ ID NO: 10 - DNA, 25 bp, Homo sapiens
actgtcaagctgcccaactgtgccc

SEQ ID NO: 11 - DNA, 23 bp, Homo sapiens
ctaccattcattgtgacttcctg

SEQ ID NO: 12 - DNA, 23 bp, Homo sapiens
gtataactgcatgtgctgctcac

SEQ ID NO: 13 - DNA, 40 bp, Artificial Sequence
  Description of Artificial Sequence  Primer
cgatttaggtgacactataggcatgaagctggcattcctc

SEQ ID NO: 14 - DNA, 42 bp, Artificial Sequence
  Description of Artificial Sequence  Primer
cgtaatacgactcactataggggtctgcagagagcagctagc